(19) United States
(12) Patent Application Publication
(10) Pub. No.: US 2022/0313983 A1
(43) Pub. Date: Oct. 6, 2022

(54) CONDUCTIVE PAD

(71) Applicant: R. G. Medical Technologies, Inc., Wixom, MI (US)

(72) Inventors: Ronald Gilbert LeTourneau, Wixom, MI (US); Gregory W. Auner, Wixom, MI (US)

(73) Assignee: R. G. Medical Technologies, Inc., Wixom, MI (US)

(21) Appl. No.: 17/707,119

(22) Filed: Mar. 29, 2022

Related U.S. Application Data
(60) Provisional application No. 63/169,984, filed on Apr. 2, 2021.

(51) Int. Cl. A61N 1/04

(52) U.S. Cl. CPC A61N 1/0492; A61N 1/0452

(57) ABSTRACT

A conductive pad includes a surface and a plurality of bumps extending from the surface. Each of the plurality of bumps includes a plurality of pores. The surface and the plurality of bumps defines respective cavities therebetween. A conductive gel is disposed in the cavities. The conductive gel is compressible through the plurality of pores.

### CROSS-REFERENCE TO RELATED APPLICATION

This application claims priority to provisional U.S. Patent Appl. No. 63/169,984, filed on Apr. 2, 2021, which is hereby incorporated by reference in its entirety.

### BACKGROUND

Conductive pads are used in the medical field to deliver electrical current to cause controlled muscle contraction that stimulates blood flow in a portion of a person's body. The conductive pads are placed on the person's skin and deliver voltage to the person's muscle. However, the voltage delivered by the pads may be insufficient to stimulate blood flow or may be overly concentrated such that the voltage causes pain and irritation to the person's skin.

### BRIEF DESCRIPTION OF THE DRAWINGS

FIG. 1 is an exploded view of an exemplary device inclusive of a housing and a pad.

FIG. 2 is a cross-sectional view along line 2-2 in FIG. 1.

FIG. 3 is a perspective view of a conductive pad connected with wires.

### DETAILED DESCRIPTION

With reference to the Figures, a conductive pad 16 is shown. The conductive pad is received on a backing 30 which in turn is fastened to an electrode 14 to form a device 10. The conductive pad 16 is in electrical communication with the electrode 14 through the backing 30. The conductive pad 16 includes a surface 18 and a plurality of bumps 20 extending from the surface 18. Each of the bumps 20 includes a plurality of pores 22. The surface 18 and the plurality of bumps 20 define respective cavities 24 therebetween. A conductive gel 26 is disposed within the cavities 24. The conductive gel 26 is compressible through the plurality of pores 22.

The conductive pad 16 may be used in conjunction with causing controlled muscle contraction that stimulates blood flow in a portion of a person's body, e.g., a leg or lower back, which may, by way of non-limiting example, reduce pain. The conductive pad 16 is pressed against the person's skin and conducts electrical current to the muscle. However, a conductive pad may concentrate voltage on the person that can result in a corona discharge to the person's skin, which may cause pain and irritation. Advantageously, the conductive gel 26 can be discharged from the plurality of bumps 20 and spread across the person's skin thereby dispersing the voltage substantially uniformly across an area of the person's skin that is covered by the conductive pad 16 which in turn reduces voltage concentrations. Dispersing the voltage applied to the person via the bumps 20 and the conductive gel 26, causes controlled muscle contraction that stimulates blood flow in a portion of the person's body, while substantially eliminating a corona discharge, which can reduce pain and irritation to the portion of the person's body where the voltage is applied. The conductive gel 26 further includes adhesive properties.

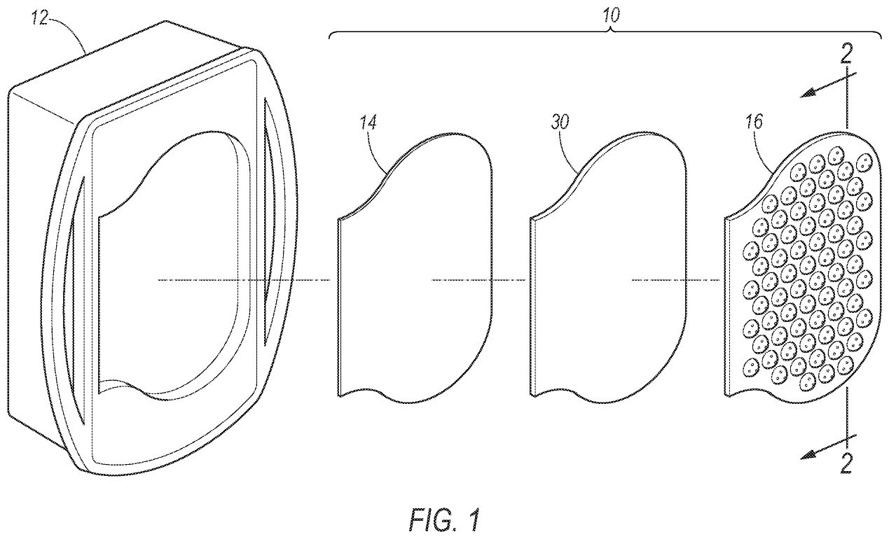

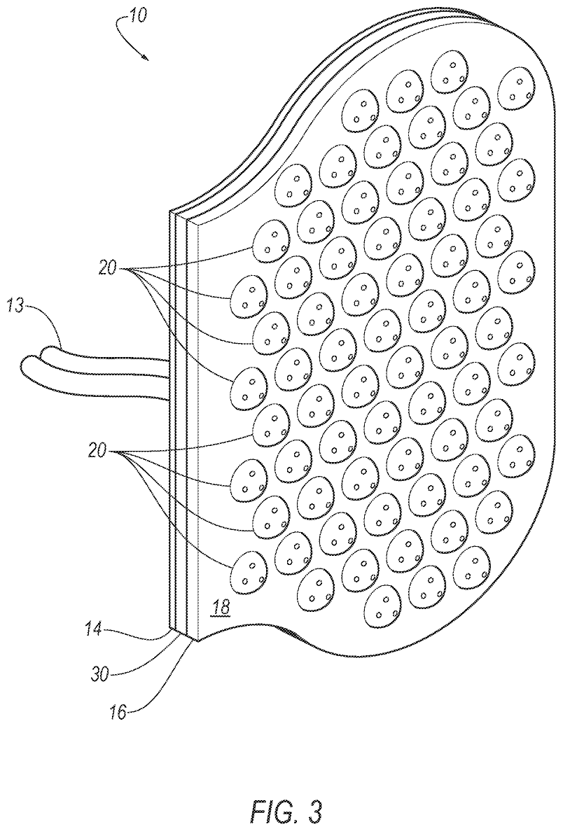

With reference to FIG. 1, the device 10 may be affixed to a person so as to rest against a portion of the person's body and to be used in conjunction with causing a controlled muscle contractions that stimulates blood flow at the portion of the person's body. Specifically, the device 10 may press against the person's body to transfer voltage to muscle tissue at the portion of the person's body, e.g., the person's lower back or leg. In one non-limiting example, the device 10 may be received in a housing 12, as shown in FIG. 1, and in another example the device 10 may be directly connected to a power source via wires 13, as shown in FIG. 3. In either example, the device 10 may be held against a person's body via the adhesive qualities of the conductive gel 26, as discussed below.

With reference to FIG. 1, the housing 12 may house various components. For example, the housing 12 may house an internal power supply (not shown), such as a battery, a controller (not shown), etc. The housing 12 may be of any suitable type and construction, e.g., a polymer, a composite, a combination of rigid materials, etc. The housing 12 may include a human-machine interface (HMI) (not numbered). The HMI includes user input devices such as buttons, switches, touchscreens, etc. The input devices may include sensors to detect user inputs. The HMI may further include output devices such as displays (including touchscreen displays), lights, etc., that output signals or data to the user.

The internal power supply provides electricity to the components housed in the housing 12. This power supply can include one or more batteries, e.g., 12-volt lithium-ion batteries, and/or one or more power networks to supply power from the batteries to the components. Since the internal power supply is housed in the housing 12, the device 10 may lack wires extending from the device 10 to an external power supply.

It is understood that the housing 12 is optional and the conductive pad 16 may be attached to the person without a housing 12. In this regard, FIG. 3 illustrates the device 10 with wires 13 connected thereto. The wires 13 extend from the electrode 14 to an external power supply (not shown) and controller (not shown).

The electrode 14 is in electrical communication with the power supply (internal or external power supply). That is, the electrode 14 receives electricity from the power supply. Specifically, the electrode 14 receives voltage from the power supply. The electrode 14 may be configured to distribute the voltage substantially uniformly across the electrode 14. For example, the electrode 14 may be formed of any suitable material that distributes electricity substantially uniformly throughout the material. As another example, the electrode 14 may have any suitable thickness that allows electricity to be substantially uniformly distributed throughout the electrode 14. The electrode 14 may be of any suitable type and construction, e.g., a conductive polymer, a metal film, a compound film such as titanium nitride, etc. The electrode 14 preferably has a same size, i.e., have a same length and width, and shape as the conductive pad 16.

In a preferred embodiment, the conductive pad 16 is formed of carbon electrical conductive silicone. Additionally, the conductive pad 16 may be coated with silver. It is understood that the conductive pad 16 may be any other suitable electrically conductive material and may include any other suitable electrically conductive coating. The conductive pad 16 can be flexible to conform to the person's skin.

The conductive pad 16 may be affixed to the backing 30. In a preferred embodiment, the conductive pad 16 is removably attached to the backing 30 using 3M™ Electrically Conductive Double-Sided Tape 9711S-150. It is understood that the conductive pad 16 may be attached to the backing 30 in any other suitable manner.

The backing 30 is disposed between the conductive pad 16 and the electrode 14. The backing 30 may be removably attached to the electrode 14. That is, the backing 30 (and the conductive pad 16) may be removed from the electrode 14. For example, the backing 30 (and the conductive pad 16) may be removed from the electrode 14 and replaced with a new backing 30 and conductive pad 16. The backing 30 may be removably attached to the electrode 14 in any suitable manner that conducts electricity, e.g., via a conductive tape, a conductive adhesive, etc. In a preferred embodiment, the backing 30 is flexible conductive silicone rubber. It is understood that the backing 30 may be any other suitable conductive material.

The backing 30 may be a same size and shape as the conductive pad 16. In other words, the backing 30, the electrode 14, and the conductive pad 16 may all be a same size and shape. In this situation, the electrode 14 may distribute voltage substantially uniformly through the backing 30 to the conductive pad 16. In other words, the voltage may be substantially uniformly distributed throughout the conductive pad 16, i.e., across the surface 18.

The surface 18 of the conductive pad 16 faces the person's skin when the device 10 is affixed to the person. The plurality of bumps 20 extend from the surface 18 upwardly relative to the surface 18. Specifically, the bumps 20 contact the person's skin when the device 10 is affixed to the person.

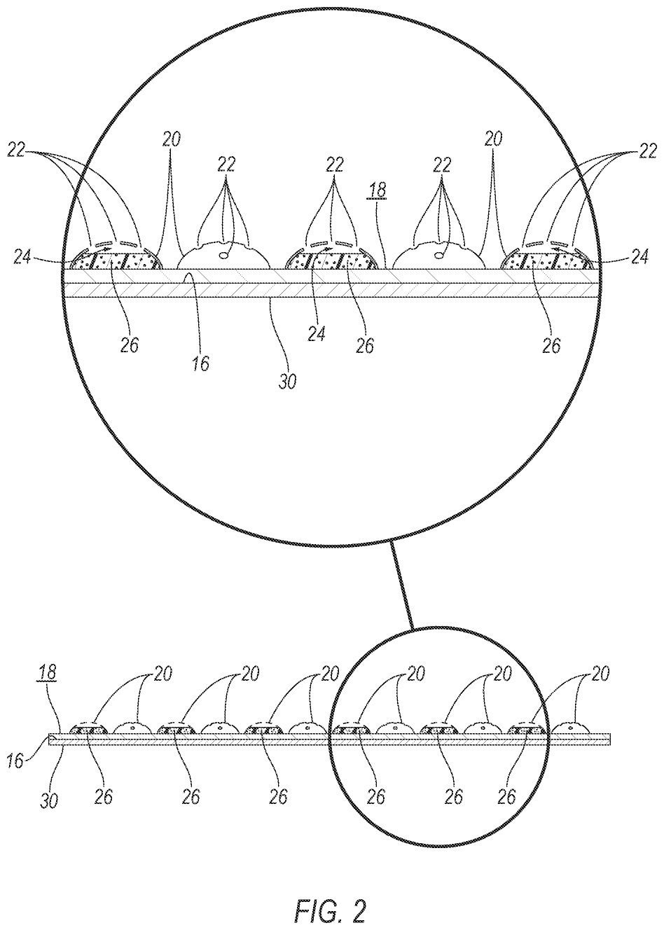

The bumps 20 may have a semi-spherical shape, as shown in FIG. 2. The bumps 20 may be convex relative to the surface 18. In a preferred embodiment, the bumps 20 have a three millimeter radius. It is understood that the bumps 20 may have any other suitable radius, e.g., two millimeters, four millimeters, five millimeters, six millimeters, etc. The bumps 20 are preferably uniform with each other, i.e., have a same size and shape.

The conductive pad 16 may include any suitable number of bumps 20. For example, the conductive pad 16 may include a number of bumps 20 that meets a density threshold. The density threshold is a measure of a number of bumps 20 per a specified area, e.g., a square centimeter, on the surface 18. The density threshold may be determined empirically, e.g., based on testing that allows for determining a density of bumps 20 that causes controlled muscle contraction that stimulates blood flow without causing corona discharge (e.g., based on a charge density of the bumps 20). In a preferred embodiment, the density threshold is between 20-25 bumps 20 per square centimeter. It is understood that the density threshold may be any other suitable density, e.g., between 15-30 bumps 20 per square centimeter.

The bumps 20 may be arranged in any suitable manner along the surface 18 such that the bumps 20 meet the density threshold. For example, the bumps 20 may be arranged in a pattern on the surface 18. In such an example, spacing between bumps 20 may be uniform, i.e., constant, between bumps, or may be repeatable with each sequence of the pattern. As another example, the bumps 20 may be arranged randomly on the surface 18. In such an example, spacing between bumps 20 may nonuniform and nonrepeatable.

With reference to FIG. 2, each bump 20 is hollow. More particularly, surface 18 and the bumps 20 define respective cavities 24 therebetween. Each bump 20 includes the plurality of pores 22, i.e., small openings, extending through the respective bump 20 into the respective cavity 24. The pores 22 can be circular. In a preferred embodiment, each bump 20 includes five pores 22. It is understood that each bump 20 may include any other suitable number of pores 22. Each bump 20 may include a same number of pores 22. The pores 22 may have any suitable size. In a preferred embodiment, the pores 22 have a 200 micron diameter. It is understood that the pores 22 may have any other suitable diameter. The pores 22 may be uniform with each other, i.e., have a same size and shape.

The pores 22 may be arranged in any suitable manner along the respective bump 20. For example, the pores 22 may be arranged in a pattern. In such an example, spacing between pores 22 may be uniform, i.e., constant, between pores 22, or may be repeatable with each sequence of the pattern. As another example, the pores 22 may be arranged randomly on the respective bump 20. In such an example, spacing between pores 22 may nonuniform and nonrepeatable.

As set forth above, the conductive gel 26 is disposed in each cavity 24, i.e., within each bump 20. In a preferred embodiment, the conductive gel 26 is Spectra® 360 12-08 Electrode Gel. It is understood that the conductive gel 26 may be any other suitable material that conducts electricity and provides a degree of adhesion. The conductive gel 26 at least partially fills the cavity 24. In a preferred embodiment, 0.05 milliliters of conductive gel 26 is disposed in each cavity 24. It is understood that any other suitable amount, i.e., volume, of conductive gel 26 may be disposed in each cavity 24, e.g., between 0.01 milliliters-0.4 milliliters. A same amount of conductive gel 26 may be disposed in each cavity 24. The volume of the conductive gel 26 in one of the cavities 24 may be smaller than a volume of that cavity 24.

The conductive gel 26 remains in the cavities 24 when the conductive pad 16 is not in contact with the person's body. The conductive gel 26 is compressible through the pores 22 of the respective bump 20. Specifically, the conductive gel 26 is forced out of the pores 22 of the respective bump 20 when the device 10 is affixed to the person. That is, the bumps 20 are compressed against the person and discharge the conductive gel 26 through the pores 22 uniformly onto the person's skin.

Further, upon being compressed through the pores 22, the conductive gel 26 can spread across the person's skin. The conductive gel 26 has sufficient cohesion properties such that as the conductive gel 26 spreads across the person's skin, the conductive gel 26 discharged from one pore 22 can stick to the conductive gel 26 discharged from another pore 22. In this situation, the conductive gel 26 may be spread substantially uniformly between the bumps 20 and the person's skin, which allows the conductive gel 26 to distribute voltage substantially uniformly to the person's skin, e.g., across an area covered by the conductive pad 16. Distributing voltage substantially uniformly to the person's skin, e.g., across the area covered by the conductive pad 16, can prevent corona discharge, thereby reducing pain and irritation to the person's skin.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. A device comprising:
a conductive pad including a surface and a plurality of bumps extending from the surface, each of the plurality of bumps including a plurality of pores;
the surface and the plurality of bumps defining respective cavities therebetween; and
a conductive gel disposed in the cavities, the conductive gel being compressible through the plurality of pores.

2. The device of claim 1, wherein the conductive gel is adhesive.

3. The device of claim 1, wherein a volume of the conductive gel in each cavity is smaller than a volume of the respective cavity.

4. The device of claim 1, further comprising a backing affixed to the conductive pad opposite the surface.

5. The device of claim 4, wherein the backing is a same shape as the conductive pad.

6. The device of claim 4, wherein the backing is flexible.

7. The device of claim 4, further comprising an electrode, wherein the backing is removably attached to the electrode.

8. The device of claim 7, wherein the backing is disposed between the conductive pad and the electrode.

9. The device of claim 1, wherein the bumps each have a semi-spherical shape.

10. The device of claim 1, wherein the bumps are arranged on the surface to have a density of at least 15 bumps per square centimeter.

11. The device of claim 1, wherein the bumps are spaced from each other.

12. The device of claim 1, wherein the bumps each have a same size and a same shape.

13. The device of claim 1, wherein the conductive pad is flexible.

14. The device of claim 1, wherein the pores are circular.

15. The device of claim 1, further comprising an electrode attached to the conductive pad and arranged to supply electricity to the conductive pad.

16. The device of claim 15, wherein the electrode is a same shape as the conductive pad.

17. The device of claim 15, wherein the electrode is configured to distribute a voltage substantially uniformly across the conductive pad.

18. The device of claim 15, further comprising a wire attached to the electrode and arranged to supply electricity to the electrode.

19. The device of claim 15, further comprising a housing, wherein the electrode and the conductive pad are received in the housing.

20. The device of claim 19, further comprising a power supply that is internal to the housing and in electrical communication with the electrode.
